(19) United States
(12) Patent Application Publication
(10) Pub. No.: US 2022/0023514 A1
(43) Pub. Date: Jan. 27, 2022

(54) FEEDING ACCESSORY AND FEEDING ASSEMBLY

(71) Applicant: THINK GREEN LIMITED, Rosedale (NZ)

(72) Inventor: Shu Ting Zhang, Jingshan City (CN)

(21) Appl. No.: 17/351,910

(22) Filed: Jun. 18, 2021

(30) Foreign Application Priority Data
Jul. 8, 2020 (CN) 202010659681.8

(51) Int. Cl. A61M 1/06

(52) U.S. Cl. CPC A61M 1/068; A61M 2205/02

(57) ABSTRACT

Provided are a feeding accessory and a feeding accessory. The feeding accessory includes a nipple cap, a nipple head, a delivery tube, and a blocking member. The nipple cap has a bowl-inverted shape. The nipple head is provided in a center of an outer arc surface of the nipple cap. One end of the delivery tube is provided in a center of an inner arc surface of the nipple cap and is in communication with the cavity of the nipple head. The blocking member is provided with a liquid conducting through hole along a thickness direction of the blocking member, The blocking member is configured to be plugged into an opening of a bottle. At least the nipple cap, the nipple head, and the blocking member are made of silicone.

### CROSS-REFERENCE TO RELATED APPLICATION(S)

This application claims priority to Chinese patent application No. 202010659681.8 filed on Jul. 8, 2020, the disclosure of which is incorporated herein by reference in its entirety.

### TECHNICAL FIELD

The present disclosure relates to the technical field of maternal and infant products, and, in particular, to a feeding accessory and a feeding assembly.

### BACKGROUND

When lactating women are in the process of breastfeeding, there may be leakage of milk from one of the breasts that is not in feeding. In order to prevent milk from being wasted, a silicone breast pump may be used for breast pumping. When the child needs to eat more milk, the milk in the silicone breast pump may be poured into other containers such as nursing bottles for secondary feeding. Besides, some women with inverted nipples cannot breastfeed directly. They usually use a silicone breast pump to extract the milk and then pour the milk in the silicone breast pump into other containers such as a nursing bottle for feeding.

It can he seen that the current silicone breast pump only has a single function of pumping and receiving milk. When breastfeeding is required, the milk in the silicone breast pump can only be poured into the bottle to achieve breastfeeding. The operation is complicated and requires more components.

### SUMMARY

Embodiments of the present disclosure provide a feeding accessory with a simple structure, which can cooperate with an open bottle body (such as a bottle body of a nursing bottle or a silicone breast pump) to realize breastfeeding, and has a wide application range.

Embodiments of the present disclosure provide a feeding component, which can realize the three functions of milk pumping, milk feeding, and milk storage.

Provided is a feeding accessory. The feeding accessory includes a nipple cap, a nipple head, a delivery tube, and a blocking member.

The nipple cap has a bowl-inverted shape.

The nipple head is provided in a center of an outer arc surface of the nipple cap, and the nipple head is provided with a cavity.

One end of the delivery tube is provided in a center of an inner arc surface of the nipple cap and is in communication with the cavity of the nipple head.

The blocking member is provided with a liquid conducting through hole along a thickness direction of the blocking member.

The liquid conducting through hole is in communication with the delivery tube, the blocking member is configured to be plugged into an opening of a bottle, and the blocking member is connected to another end of the delivery tube.

At least the nipple cap, the nipple head, and the blocking member are made of silica gel.

In an embodiment, the nipple cap, the nipple head, the delivery tube, and the blocking member are manufactured in one piece by using silicone.

In an embodiment, at least two seal rings are provided on an outer peripheral wall of the blocking member, and at least two seal rings are disposed at intervals along the thickness direction of the blocking member.

In an embodiment, two adjacent seal rings disposed at intervals form a groove surrounding the outer peripheral wall of the blocking member.

The blocking member is provided with a first side face and a second side face disposed opposite to each other.

Two ends of the liquid conducting through hole are configured to respectively extend to the first side face and the second side face, and an end of the delivery tube facing away from the nipple cap is connected to the first side face.

The second side face is provided with at least one first diversion groove, the first diversion groove extends along a radial direction of the blocking member.

One end of the first diversion groove is connected to the outer peripheral wall of the blocking member, and another end of the first diversion groove is in communication with the liquid conducting through hole.

In an embodiment, a bottom of the first diversion groove is configured to incline to an inside of the blocking member from one end of the bottom connected with the outer peripheral wall of the blocking member to another end of the bottom connected with the liquid diversion through hole.

In an embodiment, the second side face is provided with at least three first diversion grooves in an annular shape.

The blocking member is provided with an air vent extending through a thickness of the blocking member. the air vent is provided with a one-way valve, and the one-way valve is capable of delivering gas only from the first side face to the second side face.

In an embodiment, two seal rings are provided on the outer peripheral wall of the blocking member, and the two seal rings are disposed at the two ends along the thickness direction of the blocking member.

In an embodiment, at least one vent hole is provided on the nipple cap.

In an embodiment, the vent hole is configured to extend from an inner arc surface of the nipple cap to an outer arc surface of the nipple cap and incline to the blocking member, and the vent hole is in communication with an edge of the nipple cap.

In an embodiment, the thickness of the nipple cap is configured to gradually increase from a center toward an edge of the nipple cap.

In an embodiment, the blocking member is arranged at an angle to a central axis of the conveying pipe.

Provided is a feeding accessory includes the feeding accessory as above and a bottle with an opening.

In an embodiment, the bottle is a silicone breast pump, and the silicone breast pump includes a bottle body and a breast pump head which are manufactured in one piece by using silicone.

The breast pump head is tapered, a small end of the breast pump head is connected with the opening of the bottle body, and an inner side of a large end of the breast pump head is configured to be attached to a breast.

The blocking member is configured to block an internal space of the bottle body, and an edge of the nipple cap of the breastfeeding accessory is configured to be attached to an inner side wall of the breast pump head.

The beneficial effects of the present disclosure are described as follows:

By providing the breastfeeding accessory, the blocking member is plugged into the opening of the bottle, and then the breastfeeding can be realized. The breastfeeding accessory 100 may be cooperated with any bottle with an opening, such as the bottle body of a nursing bottle or silicone breast pump. The breastfeeding accessory has a wide application range. Especially after the breastfeeding accessory is cooperated with the silicone breast pump, breastfeeding is achieved without needing to pour the milk in the silicone breast pump into other containers, which is convenient for disassembly and assembly and is reliable for sealing, so as to ensure that the milk will not overflow during feeding. Besides, the silicone nipple cap with the bowl-inverted shape may imitate the women breast, which makes the baby feel the silicone nipple cap more like the women breast in the process of breastfeeding, may effectively support the baby's mouth, and improve the comfort of breastfeeding for the baby.

### BRIEF DESCRIPTION OF DRAWINGS

The present disclosure will be further described in detail based on the drawings and embodiments below

FIG. 1 is a first perspective view of a feeding accessory according to embodiments of the present disclosure.

FIG. 2 is a second perspective view of a feeding accessory according to embodiments of the present disclosure.

FIG. 3 is a perspective view of a feeding assembly according to embodiments of the present disclosure.

FIG. 4 is an exploded view of a feeding assembly according to embodiments of the present disclosure.

### DESCRIPTION OF REFERENCE NUMERALS

100 breastfeeding accessory
1 nipple cap
11 outer arc surface
12 inner arc surface
13 vent hole
14 edge
2 nipple head
3 delivery tube
4 blocking member
41 liquid conducting through hole
42 first side face
43 second side face
44 first diversion groove
45 air vent
46 one-way valve
5 seal ring
6 groove
200 silicone breast pump
7 breast pump head
8 bottle body
81 bottleneck

### DETAILED DESCRIPTION

The present disclosure will be described in further detail below in conjunction with the accompanying drawings. Apparently, the described embodiments are only some embodiments of the present disclosure, not all embodiments. Based on the embodiments of the present disclosure, all other embodiments obtained by those skilled in the art without creative work should fall within the protection scope of the present disclosure.

In the description of the present disclosure, unless otherwise expressly specified and limited, terms “ connect to”, “connected” and “fixed” should be construed in a broad sense, for example, may be securely connected, detachably connected or integrated, or may be mechanically connected or electrically connected, or may be directly connected or indirectly connected through an intermediate medium, or may be the internal communication between two elements or the interaction relationship between two elements. For those skilled in the art, specific meanings of the preceding terms in the present disclosure may be understood based on specific situations.

In the present disclosure, unless otherwise expressly specified and limited, when a first feature is described as “on” or “below” a second feature, the first feature and the second feature may be in direct contact or may be in contact via another feature between the two features instead of being in direct contact. Moreover, that the first feature is “on”, “above” or “over” the second feature includes that the first feature is right above or obliquely above the second feature or just indicates that the horizontal level of the first feature is higher than the horizontal level of the second feature. That the first feature is “under”, “below” or “beneath” the second feature includes that the first feature is right below or obliquely below the second feature or just indicates that the horizontal level of the first feature is lower than the horizontal level of the second feature.

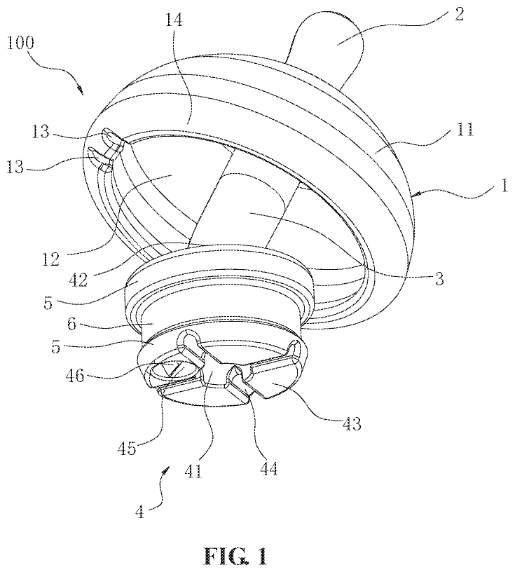

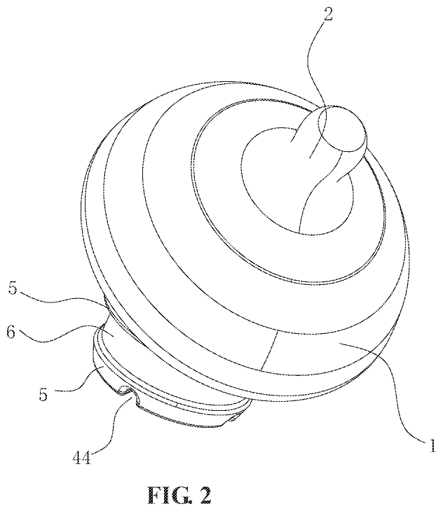

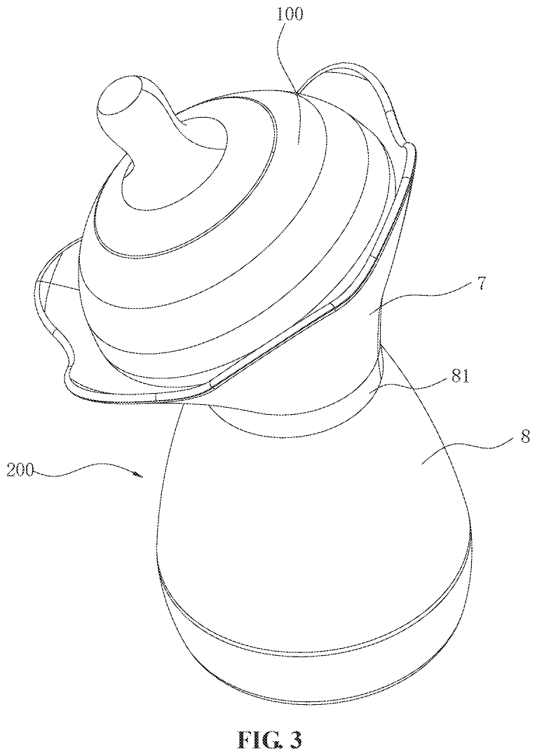

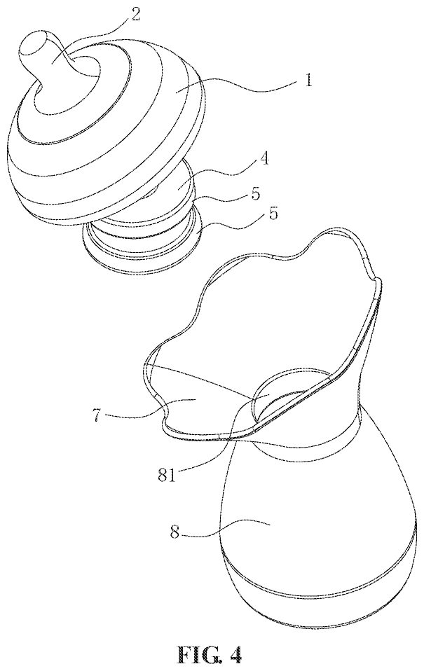

As shown in FIGS. 1 and 2 (partial reference numerals refer to FIGS. 3 and 4), the breastfeeding accessory 100 according to embodiments of the present disclosure may be used fir infant feeding. The breastfeeding accessory 100 includes a nipple cap 1, a nipple head 2, a delivery tube 3, and a blocking member 4. The nipple cap 1 has a bowl-inverted shape. The nipple head 2 is provided at the center of the outer arc surface 11 of the nipple cap 1 and is provided with a cavity. One end of the delivery tube 3 is disposed in the center of the inner arc surface 12 of the nipple cap 1 and is in communication with the cavity of the nipple head 2, and another end of the delivery tube 3 is connected to the blocking member 4. The blocking member 4 is provided with a liquid conducting through hole 41 along the thickness direction of the blocking member 4. The liquid conducting through hole 41 is in communication with the delivery tube 3, and the blocking member 4 is configured to be plugged into an opening of a bottle. At least the nipple cap 1, the nipple head 2, and the blocking member 4 are made of silicone. By setting the breastfeeding accessory 100, after the blocking member 4 of the breastfeeding accessory 100 is plugged into the opening of the bottle, the breastfeeding, can be realized. The breastfeeding accessory 100 may be cooperated with any bottle with an opening, such as the silicone breast pump 200 or a bottle body of a nursing bottle. Thus the breastfeeding accessory 100 has a wide application range. Especially after the breastfeeding accessory 100 is cooperated with the silicone breast pump 200, breastfeeding is achieved without needing to pour the milk in the silicone breast pump 200 into another container. After pumping breast milk is completed, the blocking member 4 only needs to be plugged into the opening of the silicone breast pump 200 to feed the baby, and thus the operation is very convenient. The silicone blocking member 4 can be easily plugged into the opening of the bottle, which is convenient for disassembly and assembly and is reliable for sealing, so as to ensure that the milk will not overflow during feeding. Besides, the silicone nipple cap 1 with the bowl-inverted shape may imitate the women breast, which makes the baby feel the silicone nipple cap 1 more like the women breast in the process of breastfeeding, may effectively support the baby's mouth, and improve the comfort of breastfeeding for the baby.

Embodiments of the present disclosure provide a milking assembly. The milking assembly includes breastfeeding accessory 100 and the bottle, and the blocking member 4 of the breastfeeding accessory 100 is plugged into the opening of the bottle. The bottle may be a silicone breast pump 200 as shown in FIG. 3. In the following, as an example, the bottle is the silicone breast pump 200.

As shown in FIGS. 3 and 4 (partial reference numerals follow FIGS. 1 and 2), the silicone breast pump 200 includes a bottle body 8 and a breast pump head 7, which are manufactured in one piece by using silicone. The breast pump head 7 is tapered, the small end of the breast pump head 7 is connected to the opening of the bottle body 8, the inner surface of the large end of the breast pump head 7 is in contact with the breast. The inner space of the bottle body 8 is sealed by the blocking member 4. The edge 14 of the nipple cap 1 of the breast⁻feeding accessory 100 fits with the inner side wall of the breast pump head 7.

This breastfeeding assembly has the functions of pumping, feeding, and storing milk. When it needs to pump milk, the breast pump head 7 of the silicone breast pump 200 is only attached to the breast, and the bottle body 8 is pressed to pump the milk. When it needs to store milk, the blocking member 4 of the breastfeeding accessory 100 is passed through the breast pump head 7 and plugged into the opening of the bottle body 8 (that is, in the bottleneck 81 of the bottle body 8), while the edge 14 of the nipple cap 1 is attached to the inner wall of the nipple head 7, which realizes the dust-proof and sealing effect and can preserve the milk without pouring the milk into other containers. And because of the good sealing effect, the milk will not be contaminated. When breastfeeding is needed, the baby may suck breast milk directly through the nipple head 2 on the nipple cap 1, there is no need to pour the milk into other containers during the whole process, and the operation is simple.

In this embodiment, the nipple cap 1, the nipple head 2, the delivery tube 3, and the blocking member 4 are manufactured in one piece by using silicone. The breastfeeding accessory 100 manufactured in one piece does not need to be assembled, has good structural integrity, has no the situation of small parts being lost, and can also reduce the difficulty of assembling the breastfeeding accessory 100 with the silicone breast pump 200.

In other embodiments, various components may be manufactured separately, or some two components may be made of silicone, and then combined to form the breastfeeding accessory 100 through assembly. For example, the nipple cap 1, the nipple head 2, and the delivery tube 3 are manufactured in one piece by using silicon, the blocking member 4 is made of silicon alone, and then the delivery tube 3 is inserted into the liquid conducting through hole 41 of the blocking member 4. In order to reduce the difficulty of assembling the delivery tube 3 with the blocking member 4, a threaded structure may be directly formed on the outer wall of the delivers tube 3, and a corresponding threaded structure may also be formed in the liquid conducting through hole 41, and the delivery tube 3 may be assembled with the plugging part 4 by the manner of thread connection.

Naturally, the delivery tube 3 is not limited to being made of silicone, and may also be made of other hard plastics.

In an embodiment, the cross-section of the nipple head 2 is circular or elliptical, and the diameter of the end of the nipple head 2 facing away from the nipple cap 1 is greater than the diameter of the end of the nipple head 2 connected to the nipple cap 1. This design imitates the breast, which is convenient for the baby to hold the nipple head 2 in the baby's mouth, effectively preventing the nipple head 2 from leaving the baby's mouth at will, reducing the difficulty for the baby to suck milk, and also making it easier for the baby to transform from breastfeeding to bottle-feeding.

The surface of the end of the nipple head 2 facing away from the nipple cap 1 is provided with a nipple outlet, and the nipple outlet is any one of a circular hole, a cross-shaped hole, or a Y-shaped hole. When the nipple outlet is a circular hole, the diameter of the nipple outlet is 0.5 mm˜1 mm; when the milk outlet is a cross-shaped hole, the length of the cross-shaped hole is 3 mm˜4 mm; when the milk outlet is a Y-shaped hole, the length of the hole line is 3 mm˜4 mm. The cross-shaped hole and the Y-shaped hole are capable of adjusting the milk discharge and milk speed according to the suction force, effectively preventing milk choking.

Furthermore, the inner wall of the cavity of the nipple 2 is provided with a ring of buffer groove around the center of the delivery tube 3, and the buffer groove is adjacent to the delivery tube 3. By providing the ring of buffer groove, the speed of milk in the delivery tube 3 entering into the cavity of the nipple head 2 can be effectively slowed down, so as to effectively alleviate the situation of milk choking.

In other embodiments, a buffer groove may also be spirally coiled on the cavity wall of the cavil of the nipple 2 along the length direction of the delivery tube 3. The buffer groove spirally coiled. on the cavity wall of the cavity has a more effective effect of slowing down the speed of milk, thereby effectively alleviating the situation of choking milk.

one embodiment, the maximum outer diameter of the nipple cap 1 is 75 mm, the maximum wall thickness of the nipple cap 1 is 7 mm. The nipple cap 1 is inverted bowl-shaped, which imitates a female breast.

In this embodiment, the thickness of the nipple cap 1 gradually increases from the center of the nipple cap 1 toward the edge 14 of the nipple cap 1. This design can keep the thickness of the area of the nipple cap 1 connected with the nipple head 2 thinner, and it is easier to fit with the sucking action of the baby's mouth during the process of contacting and sucking breast milk. The thickness of the nipple cap 1 gradually thickens from the center to the edge 14, which can ensure the good support effect of the nipple cap 1 and is not easy to collapse and deform as a whole. When the silicone breast pump 201) is cooperated with the breastfeeding accessory 100, the edge 14 of the nipple cap 1 is abutted against the inner wall of the breast pump head 7 of the silicone breast pump 200, and thus the non-deformable nipple cap 1 can also cover the mouth of the breast pump head 7. which has the dust-proof effect.

At least one vent hole 13 is provided on the nipple cap 1. When the breastfeeding accessory 100 is in use, a suction action is generated, and the milk in the silicone breast pump 200 may he output normally. When the suction action occurs, the air in the silicone breast pump 200 is sucked away. After the breastfeeding accessory 100 is assembled with the silicone breast pump 200, the blocking member 4 is occluded by the nipple cap 1. under this situation, in order to supplement fresh air, the vent hole 13 is provided on the nipple cap 1 to continuously supply fresh air into the nipple cap 1.

In the embodiment, the vent hole 13 is configured to extend from the inner arc surface 12 of the nipple cap 1 to the outer arc surface 11 of the nipple cap 1 and incline to the blocking member 4, and the vent hole 13 is in communication with the edge 14 of the nipple cap 1. The obliquely vent hole 13 enable the inner arc surface 12 of the nipple cap 1 to have no corner structure, to reduce the resistance of air entering the nipple cap 1 from the vent hole 13 and speed up the inhalation efficiency; The vent hole 13 on the edge 14 of the nipple cap 1 may be cooperate with the second diversion groove (not shown in the FIGS) on the inner side wall of the breast pump head 7 of the silicone breast pump 200 to accelerate air circulation. At the same time, there is no need to open a larger vent hole 13 on the nipple cap 1, that is, the strength of the nipple cap 1 will not be reduced.

In an embodiment, the length of the vent hole 13 is 5 mm, the width of the vent hole 13 is 7 mm (that is, the thickness of the nipple cap 1), and the height of the vent hole 13 is 4 mm.

The edge 14 of the nipple cap 1 is provided with two vent holes 13.

The delivery tube 3 has a cylindrical shape with an inner diameter of 10 mm to 12 mm. The delivery tube 3 is the main part for connecting the nipple head 2 with the blocking member 4 and is also the main channel for breast milk. On the market, the nursing bottle products are directly connected to the nipple. When they are in use, the nursing bottle needs to be inverted to cause the milk to flow to the nipple under the gravity of the gravity center. When the milk flows to the nipple, with the influence of the airflow in the bottle, the milk spraying effect is easy to form at the milk outlet, leading to milk choking for the baby. Especially at the beginning of feeding, the weight of milk is concentrated in the inner pocket of the nipple. The pressure of gravity on the baby's face and mouth may easily cause the baby's face and mouth to feel tired at the beginning of feeding. In contrast, The delivery tube 3 of the embodiments of the disclosure may cause the milk to flow in an equal amount and at a constant speed. The milk flowing in an equal amount and at a constant speed of the delivery tube 3 is combined with the drainage and buffering effect of the cross-shaped hole or Y-shaped hole on the nipple head 2 to effectively slow down and control the milk flow rate, to avoid the above-mentioned milk spraying effect, and to reduce the situation of baby's milk choking.

In one embodiment, at least two seal rings 5 are provided on the outer peripheral wall of the blocking member 4, and at least two seal rings 5 are disposed at intervals along the thickness direction of the blocking member 4. Two adjacent seal rings 5 disposed at intervals form a groove 6 surrounding the outer peripheral wall of the blocking member 4. By providing the sealing rings 5, the difficulty of plugging of the blocking member 4 into the opening of the bottle body can be reduced, and the sealing performance is good, especially when at least two sealing rings 5 are provided, the sealing effect is better. By providing the groove 6, the space may be provided for the seal ring 5 that is squeezed and deformed.

In this embodiment, the outer peripheral wall of the blocking member 4 is provided with two sealing rings 5, and the two sealing rings 5 are respectively disposed at both ends of the blocking member 4 in the thickness direction of the blocking member 4.

Furthermore, the bottle body 8 of the silicone breast pump 200 is provided with a bottleneck 81, the upper end of the bottleneck 81 is provided with the opening, the lower end of the bottleneck 81 is in communication with the inner cavity of the bottle body 8. When the blocking member 4 is plugged into the bottle body 8, the two sealing rings 5 of the blocking member 4 are respectively located at the two ends of the bottleneck 81 to form a buckle effect, and the groove 6 on the outer periphery of the blocking member 4 is in contact with the inner wall of the bottleneck 81 to generate traction, so that the blocking member 4 is not easily pulled out. Under the effect of the double force, the blocking member 4 can realize a double sealing effect after being installed on the silicone breast pump 200, so that the milk can be steadily stored in the bottle body 8 of the silicone breast pump 200 during the breastfeeding process.

In an embodiment, the blocking member 4 is provided with a first side face 42 and a second side face 43 opposite to each other, and two ends of the liquid conducting through hole 41 respectively extend to the first side face 42 and the second side face 43, the end of the delivery tube 3 facing away from the nipple cap 1 is connected to the first side face 42. The diameter of the seal ring 5 adjacent to the first side face 42 is greater than the diameter of the seal ring 5 adjacent to the second side face 43. The blocking member 4 is cylindrical. The above design can make the structure composed of the blocking member 4 and the seal ring 5 have a structure with a large end and a small end, which is convenient for the structure to be plugged into the opening of the bottle body 8 (that is, in the bottleneck 81). In an embodiment, the diameter of the sealing ring 5 adjacent to the first side face 42 is 40 mm, and the thickness of the sealing ring 5 adjacent to the first side face 42 is 7 mm. The diameter of the seal ring 5 adjacent to the second side face 43 is 36 mm, and the thickness of the seal ring 5 adjacent to the second side face 43 is 6 mm. The outer diameter of the blocking member 4 is 34 mm. The thickness of the blocking member 4 is 12 mm.

In an embodiment, the second side face 43 is provided with at least one first diversion groove 44. the first diversion groove 44 extends along the radial direction of the blocking member 4, and one end of the first diversion groove 44 is connected to the outer peripheral wall of the blocking member 4, and another end of the first diversion groove 44 is in communication with the liquid conducting through hole 41. By providing the first diversion groove 44, the milk in the bottle body 8 of the silicone breast pump 200 can be smoothly introduced into the liquid conducting through hole 41 of the blocking member 4, at the same time, the first diversion groove 44 also has the buffering effect for the milk. Besides, the first diversion groove 44 can also prevent milk from accumulating at the blocking member 4, so that all the milk can be led out into the delivery tube 3.

In this embodiment, the bottom of the first diversion groove 44 inclines to the inside of the blocking member 4 from one end of the bottom connected with the outer peripheral wall of the blocking member 4 to another end of the bottom connected with the liquid conducting through hole 41. The first diversion groove 44 which is obliquely provided has a better diversion effect and realizes the effect that the milk al the edge converges toward the middle,

In an embodiment, the second side face 43 is provided with at least three first diversion grooves 44 in an annular shape.

In one embodiment, the blocking member 4 is provided with an vent 45 extending through the thickness of the blocking member 4. The air vent 45 is provided with a one-way valve 46, and the one-way valve 46 is capable of delivering gas only from the first side face 42 to the second side face 43. By providing the air vent 45 and the one-way valve 46, it can he ensured that the silicone breast pump 200 maintains the balance of the air pressure inside and outside the bottle body 8 during the feeding process to prevent the bottle body 8 from deforming. While providing the one-way valve 46 can prevent the milk from passing through the air vent 45 and overflowing to the outside of the bottle body 8 during the feeding process.

the blocking member 4 is arranged at an angle to the central axis of the delivery tube 3. This arrangement enables the nipple cap 1 to incline at a certain angle relative to the blocking member 4, thereby ensuring that the nipple cap 1 can be attached to the inner wall of the breast pump head 7 of the silicone breast pump 200.

It is understandable that the breastfeeding accessory 100 of the present disclosure is not limited to cooperating with the silicone breast pump 200 to realize breastfeeding, and may also directly cooperate with the bottle body 8 of an existing nursing bottle to realize breastfeeding.

In the description of the disclosure, it should be understood that the term “up” and other orientations or positional relationships are based on the orientations or positional relationships shown in the drawings, and are only for ease of description and simplified operations, and do not indicate or imply the device or element referred to must have a specific orientation, be constructed and operated in a specific orientation, and therefore cannot he understood as a limitation to the present disclosure.

In the description of this specification, the description regarding the term “an embodiment” or the like means that a specific feature, structure, material, or characteristic of the embodiment is included in at least one embodiment or example of the present disclosure. In this specification, the schematic representations of the above-mentioned terms do not necessarily refer to the same embodiment.

Besides, it should be understood that although this specification is described in accordance with the implementation modes, not each implementation mode only includes an independent technical solution. This narration in the specification is only for clarity, and those skilled in the art should regard the specification as a whole The technical solutions in the various embodiments can also be appropriately combined to form other implementations that can be understood by those skilled in the art.

The technical principle of the present invention has been described above in conjunction with specific embodiments. These descriptions are only for explaining the principle of the present disclosure, and cannot be construed as limiting the protection scope of the present invention in any way. Based on the explanation here, those skilled in the art can think of other specific implementation manners of the present disclosure without creative work, and these manners will fall within the protection scope of the present disclosure.

1. A feeding accessory, comprising:
a nipple cap, wherein the nipple cap has a bowl-inverted shape;
a nipple head, wherein the nipple head is provided in a center of an outer arc surface of the nipple cap, and the nipple head is provided with a cavity;
a delivery tube, wherein one end of the delivery tube is provided in a center of an inner arc surface of the nipple cap and is in communication with the cavity of the nipple head; and
a blocking member, wherein the blocking member is provided with a liquid conducting through hole along a thickness direction of the blocking member, the liquid conducting through hole is in communication with the delivery tube, the blocking member is configured be plugged into an opening of a bottle, and the blocking member is connected to another end of the delivery tube;
wherein at least the nipple cap, the nipple head, and the blocking member are made of silicone.

2. The feeding accessory according to claim 1, wherein the nipple cap, the nipple head, the delivery tube, and the blocking member are manufactured in one piece by using silicone.

3. The feeding accessory according to claim 1, wherein at least two seal rings are provided on an outer peripheral wall of the blocking member, and the at least two seal rings are disposed at intervals along the thickness direction of the blocking member;
two adjacent seal rings disposed at intervals form a groove surrounding the outer peripheral wall of the blocking member.

4. The feeding accessory according to claim 3, wherein the blocking member is provided with a first side face and a second side face disposed opposite to each other, and two ends of the liquid conducting through hole are configured to respectively extend to the first side face and the second side face; an end of the delivery tube facing away from the nipple cap is connected to the first side face;
the second side face is provided with at least one first diversion groove, the first diversion groove extends along a radial direction of the blocking member;
one end of the first diversion groove is connected to the outer peripheral wall of the blocking member, and another end of the first diversion groove is in communication with the liquid conducting through hole.

5. The feeding accessory according to claim 4, wherein a bottom of the first diversion groove is configured to incline to an inside of the blocking member from one end of the bottom connected with the outer peripheral wall of the blocking member to another end of the bottom connected with the liquid conducting through hole.

6. The feeding accessory according to claim 4, wherein the second side face is provided with at least three first diversion grooves in an annular shape.

7. The feeding accessory according to claim 1, wherein the blocking member is provided with an air vent extending through a thickness of the blocking member. The air vent is provided with a one-way valve, and the one-way valve is capable of delivering gas only from the first side face to the second side face.

8. The feeding accessory according to claim 3, wherein two seal rings are provided on the outer peripheral wall of the blocking member, and the two seal rings are disposed at the two ends along the thickness direction of the blocking member.

9. The feeding accessory according to claim 1, wherein at least one vent hole is provided on the nipple cap.

10. The feeding accessory according to claim 9, wherein the vent hole is configured to extend from an inner arc surface of the nipple cap to an outer arc surface of the nipple cap and incline to the blocking member, and the vent hole is in communication with an edge of the nipple cap.

11. The feeding accessory according to claim 1, wherein the thickness of the nipple cap is configured to gradually increase from a center of the nipple cap toward an edge of the nipple cap.

12. The feeding accessory according to claim 11, wherein the blocking member is arranged at an angle to a central axis of the conveying pipe.

13. A feeding accessory, comprising:
a bottle with an opening; and
a feeding accessory;
wherein the feeding accessory comprises:
a nipple cap, wherein the cripple cap has a howl-inverted shape;
a nipple head, wherein the nipple head is provided in a center of an outer arc surface of the nipple cap, and the nipple head is provided with a cavity;
a delivery tube, wherein one end of the delivery tube is provided in a center of an inner arc surface of the nipple cap and is in communication with the cavity of the nipple head; and
a blocking member, wherein the blocking member is provided with a liquid conducting through hole along a thickness direction of the blocking member, the liquid conducting through hole is in communication with the delivery tube, the blocking member is configured be plugged into the opening of the bottle, and the blocking member is connected to another end of the delivery tube;
wherein at least the nipple cap, the nipple head, and the blocking member are made of silicone;
the blocking member is configured to be plugged into the opening of the bottle body.

14. The feeding accessory according to any one of claims 13, wherein the bottle is a silicone breast pump, the silicone breast pump comprises a bottle body and a breast pump head which are manufactured in one piece by using silicone, the breast pump head is tapered, and a small end of the breast pump head is connected with the opening of the bottle body, an inner side of a large end of the breast pump head is configured to be attached to a breast, the blocking member is configured to block an internal space of the bottle body, and an edge of the nipple cap of the breastfeeding accessory is configured to be attached to an inner side wall of the breast pump head.
